(19) United States
(12) Patent Application Publication
(10) Pub. No.: US 2022/0211604 A1
(43) Pub. Date: Jul. 7, 2022

(54) ANTIMICROBIAL COMPOSITION FOR SELECTIVE LYSIS OF S. HOMINIS BACTERIA

(71) Applicant: Conopco, Inc., d/b/a UNILEVER, Englewood Cliffs, NJ (US)

(72) Inventors: Sayandip Mukherjee, Bangalore (IN); Sandip Bhanudas Pathak, Bangalore (IN)

(21) Appl. No.: 17/602,180

(22) PCT Filed: Apr. 2, 2020
(86) PCT No.: PCT/EP2020/059346

§ 371 Date: Oct. 7, 2021

(30) Foreign Application Priority Data
Apr. 9, 2019 (EP) 19168025.5

(51) Int. Cl. A61K 8/66; C12N 9/36; A61Q 15/00

(52) U.S. Cl. CPC A61K 8/66; C12N 9/2462; A61Q 13/00; C12Y 302/01017; A61K 2800/77; A61Q 15/00

(57) ABSTRACT

The present invention relates to a method and a composition to prevent or treat malodour, especially present in human axilla by selective lysis of S. hominis bacteria. The method comprises treating skin with an endolysin derived from a Staphylococcus hominis phage.

### FIELD OF THE INVENTION

The present invention relates to a method and a composition to prevent or treat malodour, especially present in human axilla by selective lysis of S. hominis bacteria. The method comprises treating skin with an endolysin derived from a Staphylococcus hominis bacteriophage.

### BACKGROUND OF THE INVENTION

In humans, the skin of underarm provides a unique niche for bacteria. Through the secretions of various glands that open onto skin, the environment is nutrient rich and hosts a unique microbial community. In humans, the link between axillary apocrine gland secretions, underarm bacteria and body odour have been long established. Culture-based studies on this relationship found the axillary microbiota to be dominated by Staphylococcus, Corynebacterium and Propionibacterium species. More recent culture-independent studies have confirmed the presence of these genera and additionally, indicated the presence of Gram-positive anaerobic cocci (GPAC) belonging to Anaerococcus and Peptoniphilus genera. The primary reason for these studies was to identify target bacterial species responsible for axillary malodour and thereby design strategies to control it for providing the axillary malodour reduction benefit.

Human body odour contains several chemicals, but the most pungent and recognisable are thioalcohols. These molecules are created through a series of chemical reactions that start with an odourless precursor, a compound produced in glands located in the armpits. Popular theory has it that a type of bacteria called Staphylococcus hominis (S. hominis) takes in these molecules and transforms them into thioalcohols which give the body the malodour.

The present inventors in seeking to solve the malodour problem believe that controlling the S. hominis metabolism of thioalcohol or controlling the bacterial population in underarm are important for providing end benefit. It is possible to completely eradicate or minimize all the bacteria on the skin through the use of agents like alcohol or other broad-spectrum antimicrobials. However, the present inventors wish to maintain the natural microbiome on the skin and only selectively target the desired species of bacteria which causes the production of the malodour molecules to obtain the desired results. Hence, they embarked upon exploring the possibility of bacterial control by selectively targeting S. hominis in underarm for malodour reduction while trying to keep the microbiome balance.

Body malodour has been tackled in many ways. Some of these approaches are use of perfumes to mask the malodour but this approach has benefit only for a limited time. Anti-perspirant compositions are also available but they block the sweat glands thereby depriving the body of a mechanism to excrete undesirable chemicals through sweat. Broad spectrum antimicrobial agents have also been used but they interfere with the microbiome balance on the skin. In contrast, the present approach to only target the specific microorganism responsible for creating the malodour (to the exclusion of other microorganisms) thereby not interfering with any other necessary bodily functions, found appeal with the present inventors.

The present inventors thus started working towards providing “natural” solutions to solving the problem of malodour. They took up the approach of testing new actives that kill or selectively inhibit growth of S. hominis to the exclusion of other organisms like S. epidermidis, E. coli and S. aureus and experimented with the same. They finally hit up on a Staphylococcal phage whose natural host is S. hominis and endolysins were found to selectively target S. hominis and inactivate it for ensuring control of malodour.

WO08001342 relates to the field of cloning of recombinant lysin from a staphylococcal bacteriophage and more particularly to the use of recombinant staphylococcal lysin (LysK) cloned from staphylococcal bacteriophage K and fractions thereof as an antimicrobial agent for killing a wide range of staphylococci in addition to using it for diagnostic applications. The lysin here it not very specific in targeting the bacteria of interest as it inhibits a wide range of organisms.

WO17046021 relates to the field of medicine, specifically to the field of treatment of conditions associated with Staphylococcus infection. The invention relates to a novel endolysin polypeptide specifically targeting a bacterial Staphylococcus cell. The invention further relates to said endolysin polypeptide for medical use, preferably for treating an individual suffering from a condition associated with Staphylococcus infection. Again the method here includes taking lysin from phages of various Staphylococcus bacteria and using the pool to claim treating Staphylococcus infection. The specificity of treating S. hominis with endolysin from S. hominis phage is not disclosed.

It is thus an object of the present invention to provide for selective kill or inhibition of S. hominis to tackle body malodour.

It is another object of the present invention to provide for tackling bodily malodour while maintaining microbiome balance on the skin.

### SUMMARY OF THE INVENTION

According to the first aspect of the present invention there is provided an antimicrobial composition for specifically targeting S. hominis bacteria comprising
(i) S. hominis phage-derived endolysins or nucleic acid molecules encoding the same; and
(ii) a topically acceptable carrier.

According to another aspect of the present invention there is provided a method of controlling or eradicating S. hominis from skin comprising the step of applying the composition of the first aspect on to a desired skin surface.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated.

Except in the operating and comparative examples, or where otherwise explicitly indicated, all numbers in this description indicating amounts of material or conditions of reaction, physical properties of materials and/or use are to be understood as modified by the word “about”. Unless specified otherwise, numerical ranges expressed in the format “from x to y” are understood to include x and y. When for a specific feature multiple preferred ranges are described in the format “from x to y”, it is understood that all ranges combining the different endpoints are also contemplated.

The composition as per this invention could be in the form of a leave-on or wash-off format for delivering selective malodour benefit to topical areas e.g. skin and/or hair of mammals, especially humans. Such a composition includes any product applied to a human body for also improving appearance, cleansing, or general aesthetics. The composition of the present invention may be delivered with a topically acceptable carrier which could be an anhydrous base, liquid, lotion, cream, foam, scrub, gel, emulsion or a propellant. “Skin” as used herein is meant to include skin on any part of the body (e.g., neck, chest, back, arms, underarms, hands, legs, buttocks and scalp) especially the underarm. It is especially useful for reduction of malodour from the underarm (or axilla) or any other part of the body where malodour is generated.

The composition as per the present invention comprises S. hominis i.e. Staphylococcus hominis phage-derived endolysins e.g. STB12 or nucleic acid molecules encoding the same. The composition is especially useful for selective lysis of S. hominis bacteria. By selective lysis, as per this invention, is meant that there is differential kill, with preference for S. hominis over other species of bacteria e.g. S. epidermidis, S. aureus etc. In a contact kill assay, the differential kill has been found to be quantified by observing log kill of S. hominis to be higher than 1.0, preferably higher than 1.6, as compared to other bacteria which are found to be generally less than 1.6 log kill, and in most cases to be less than 1.0 log kill.

The present inventors looked at various approaches for inhibiting S. hominis and arrived at the application of bacteriophages and bacteriophage-derived enzymes towards this end. A group of phage-derived enzymes are peptidoglycan (PG) hydrolases known as endolysins. Endolysins are novel muralytic hydrolases encoded by double stranded DNA phages which degrade the PG layer of the bacterial cell wall thereby allowing the progeny phages to escape in the late phase of the infection cycle. Purified endolysins when exposed to PG externally can cause “lysis from outside”. Based on their antimicrobial properties (extraordinary substrate specificity and high activity when added externally) endolysins from phages infecting Gram-positive pathogens has been the motivation and hypothesis for the present inventors as one element of the composition of the present invention.

Thus, it is useful that the endolysin that is included is a recombinant form of S. hominis phage endolysin. This endolysin is preferably cloned from endolysin gene sequence (Gene ID: 26066873; 1467 nucleotide base pair long, 489 amino acids, protein ID: YP_009130751.1) from Staphylococcal phage STB12 (GenBank: NC_020490.2) which is codon optimized for expression in E. coli and cloned into commercially available pET303/CT-His expression vector.

The nucleotide sequence of endolysin which is especially useful is as per the sequence ID SEQ ID1 which is listed below:

ATGCTGATGACCCGCAAACAGGCGGAAAAATGGCTGGATAACAGCGAAGG
CCGCCAGTATAACGCGGATGGCTATTATGGCTTTCAGTGCTATGATTATA
GCAAAATGTATTTTTATGTGGTGACCGGCGAATGGATTGGCGGCCTGAAA
GCGAGCAACATTCCGTTTGATAACAAAGCGAAAATTGAAAAATATGCGAC
CATTATTAAAAACTATGATAGCTTTCTGCCGCAGAAAGGCGATATTGTGT
GCTTTCCGAACAAATATGGCGGCGGCTATGGCCATACCGCGGTGGTGACC
AAAGCGACCCTGACCCAGTTTGAAGTGCTGGAACAGAACTGGTTTGGCAA
CGGCTGGACCGATGGCGTGGTGAAACCGGGCTGGGGCCCGGAAACCGTGA
GCCGCCGCTGGCATTATTATGATAACCCGATGTATTTTATTCGCTTTAAC
TTTCCGAAAAACGTGAACGTGGTGAAAAAAGCGAAACGCAAACTGAGCAG
CAACAAAGCGAGCGGCCAGATTAAACGCAAAAAAATTATGATTGTGGCGG
GCCATGGCTATAACGATCCGGGCGCGGTGGGCAACGGCACCAACGAACGC
GATTTTATTCGCAAAAACCTGACCCCGAAAATTGCGAACTATCTGCGCAA
AACCGGCCATGAAGTGGCGCTGTATGGCGGCAGCAGCCAGAGCCAGGATA
TGTATCAGGATACCGCGTATGGCGTGCGCGTGGGCAACAAACGCGATTAT
GGCATGTATTGGGTGAACAAACAGAACTATGATCTGATTGTGGAATTTCA
TCTGGATGCGGCGGGCGCGAGCGCGAGCGGCGGCCATGTGATTATTAGCA
GCGCGTTTAACGCGGATAGCATTGATAAAGATATTCAGGAAGTGATTAAA
GAAAACCTGGGCCAGATTCGCGGCATTACCAAACGCAGCGATCTGCTGCA
TGCGAACGTGAGCGCGGAAATTAACATGAACTATCGCCTGGCGGAACTGG
GCTTTATTACCAACAAAGAAGATATGGATTGGATTAAAAAAAACAGCGAT
AAATATGCGAAACTGATTGCGGGCGCGATTCATGGCAGCCCGATTGGCGG
CGTGGTGGCGAGCAAAAAAAAAAGCAGCAGCAAAAAACTGAACGTGCCGA
AAACCATTCCGAGCGGCTATAAACTGAACAACAAAGGCGTGCCGTATAAA
AAAGAAAAAAGCCGCTATACCGTGACCACCATTAAAGGCAACAACGTGCG
CACCACCTATAGCGATAAAAGCGAAATTACCGGCACCCTGCCGAACGGCG
AAGAAATTATTTATGATGGCGCGTTTGCGGTGAACGGCTATCGCTGGATT
ACCTATCTGAACAACGATCTGCAGCGCCGCTATATTGCGACCGGCGAAAT
TGATGAAAACGGCAAACGCACCAGCAGCTATGGCAAATTTAGCCGCGTGC
ATCATCATCATCATCAT

An especially preferred aspect relates to the endolysin where the stop codon was removed from the 3′ end of the gene to accommodate a 6× Histidine tag.

It is also possible that the nucleic acid molecules of the endolysin for optional inclusion in the composition of the invention comprise fragments, variants and fusions of the endolysin which are capable of specifically binding to and/or lysing cells of S. hominis.

The present inventors have, by way of the present invention explored an enzybiotic approach for targeted reduction of S. hominis bacteria mainly responsible for axillary malodour using bacteriophage derived endolysin isolated from STB12 temperate bacteriophage. They find that these phages have specific lytic activity against S. hominis and not against other Staphylococcus species. The full genome sequence of STB12 phage is available in Genbank (https://www.ncbi.nlm.nih.gov/nuccore/NC_020490.2). Here, the inventors have used the endolysin sequence from the deposited genome, codon optimised the gene for efficient expression in E. coli. The codon optimization was done using Geneart tool (Thermofisher scientific) for E. coli expression. The cloning was done in pET 303 expression system with 6× His tag under T7 promotor. The pET303 plasmid containing codon optimised STB12 endolysin gene was transformed into chemically competent Shuffle T7 cells (NEB. USA). The overexpressed protein was purified from inclusion bodies followed by affinity chromatography purification using NiNTA chromatography columns as per the protocol standardised in lab. The purified protein was refolded by dialysis under gradient of urea and final elution was performed in HEPES buffer (pH 7.0). The purified protein was tested in Turbidity reduction assay and Contact kill assay for specificity and efficacy against S. hominis and S. epidermidis strains.

Without wishing to be bound by theory, the present inventors believe that the specificity of the Staphylococcal StB12 phage derived endolysin in S. hominis bacteria is due to the modular nature of the endolysin which allows it to selectively target the natural host of the bacteriophage from which it is derived. All endolysins derived from bacteriophages infecting Gram-positive bacteria consist of a single or multiple N-terminal enzymatic activity domain/s (EAD/s) and a single C-terminal cell wall binding domain (CBD). While the EAD is responsible for enzymatic hydrolysis of defined bonds in the peptidoglycan, the CBD confers upon the endolysin the ability to specifically attach to selected moieties in the cell wall (including secondary cell wall polysachharides and proteins such as teichoic acid).

The composition of the invention preferably includes a topically acceptable carrier. Preferred topically acceptable carrier may comprise an anhydrous base, a gel, a lotion, a cream or an emulsion. The composition could be delivered in a stick form and through a roll-on device or using a propellant containing aerosol can.

The composition may be delivered when the topically acceptable carrier is anhydrous. By an anhydrous carrier is meant that water content in the composition is less than 5wt %, preferably less than 2 wt %, more preferably less than 1 wt % and optimally absent from the composition. To enable this, the anhydrous carrier preferably comprises a silicone compound, an alcohol or a wax. The alcohol, when used, could be a low boiling (C2-C4) alcohol or a polyhydric alcohol, preferably a polyhydric alcohol.

The pH of the composition is preferably higher than 3.5 more preferably in the range of 4 to 7. The pH of the composition of the invention is measured using the following procedure:

Equal volumes of the composition and model ionic sweat (pH 6.1) are mixed, and the pH value is measured using an accurate range pH test paper.

The composition of the invention preferably comprises a polyhydric alcohol. Polyhydric alcohol is also referred to in short as polyol. A polyhydric alcohol as per the present invention is a compound having two or more hydroxyl groups. Suitable class of polyhydric alcohols that may be included in the composition of the invention are monomeric polyols, polyalkylene glycols or sugars. Preferred monomeric polyols are glycol; alkylene glycol e.g. propylene glycol; glycerol; or xylitol, more preferably propylene glycol.

Suitable polyalkylene glycols are polyethylene glycol or polypropylene glycol. Sugars for inclusion in the invention could be monomeric, dimeric, trimeric or of the polymeric form. Preferred sugars include glucose, fructose, mannose, sucrose, threitol, erythritol, sorbitol, mannitol, galactitol, adonitol, dextran, or cyclodextrin. Of these the more preferred sugars are glucose, fructose, sucrose, sorbitol, mannitol, adonitol, dextran, or cyclodextrin.

Other components commonly included in conventional compositions may also be incorporated in the composition of the present invention. Such components include skin care agents such as emollients, humectants and skin barrier promoters; skin appearance modifiers such as skin lightening agents and skin smoothing agents; anti-microbial agents, in particular organic anti-microbial agents, and preservatives.

The composition of the invention can be applied cosmetically and topically to the skin, broadly speaking, by one of two methods. Some consumers prefer one method and some others, the other method. In one method, sometimes called a contact method, a composition is wiped across the surface of the skin, depositing a fraction of the composition as it passes. In the second method, sometimes called the non-contact method, the composition is sprayed from a dispenser held proximate to the skin, often in an area of about 10 to 20 cm². The spray can be developed by mechanical means of generating pressure on the contents of the dispenser, such as a pump or a squeezable sidewall or by internally generated pressure arising from a fraction of a liquefied propellant volatilising, the dispenser commonly being called an aerosol.

There are broadly speaking two classes of contact compositions, one of which is liquid and usually applied using a roll-on dispenser or possibly absorbed into or onto a wipe, and in the second of which the antiperspirant active is distributed within a carrier liquid that forms a continuous phase that has been gelled. In one variation, the carrier fluid comprises a solvent for the antiperspirant and in a second variation, the antiperspirant remains a particulate solid that is suspended in an oil, usually a blend of oils.

### Stick or Soft Solid Compositions

Many different materials have been proposed as gellant for a continuous oil phase, including waxes, small molecule gelling agents and polymers. They each have their advantages and of them, one of the most popular class of gellant has comprised waxes, partly at least due to their ready availability and ease of processing, including in particular linear fatty alcohol wax gellants. A gelled antiperspirant composition is applied topically to skin by wiping it across and in contact with the skin, thereby depositing on the skin a thin film.

The nature of the film depends to a significant extent on the gellant that is employed. Although wax fatty alcohols have been employed as gellant for many years, and are effective for the purpose of gelling, the resultant product is rather ineffective at improving the visual appearance of skin, and in particular underarm skin, to which the composition has been applied. This problem has been solved by including ameliorating materials for example, di or polyhydric humectants and/or a triglyceride oil.

### Roll-On

Liquid compositions that are applicable from a roll-on broadly speaking can be divided into two classes, namely those in which an antiperspirant active is suspended in a hydrophobic carrier, such as a volatile silicone and those in which the antiperspirant active is dissolved in a carrier liquid. The latter has proven to be more popular. There are mainly two sorts of dissolving carrier liquid, namely carriers that are predominantly alcoholic, which is to say the greater part of the dissolving carrier fluid comprises ethanol and the second class in which the carrier liquid is mainly water. The former was very popular because ethanol is a mild bactericide in its own right, but its popularity waned because it stings, especially if the surface onto which the composition has been applied has been damaged or cut, such as can easily arise during shaving or other de-hairing operations.

The second class of formulations that is an alternative to alcoholic formulations comprise a dispersion of water-insoluble or very poorly water soluble ingredients in an aqueous solution of the antiperspirant. Herein, such compositions will be called emulsions. Antiperspirant roll-on emulsions commonly comprise one or more emulsifiers to maintain a distribution of the water-soluble ingredients.

### Aerosol Compositions

The composition of the invention may be delivered through an aerosol composition which comprises a propellant in addition to the other ingredients described hereinabove. Commonly, the propellant is employed in a weight ratio to the base formulation of from 95:5 to 5:95. Depending on the propellant, in such aerosol compositions the ratio of propellant to base formulation is normally at least 20:80, generally at least 30:70, particularly at least 40:60, and in many formulations, the weight ratio is from 90:10 to 50:50. A ratio range of from 70:30 to 90:10 is sometimes preferred.

Propellants herein generally are one of three classes; i) low boiling point gasses liquifided by compression, ii) volatile ethers and iii) compressed non-oxidising gases.

Class i) is conveniently a low boiling point material, typically boiling below −5° C., and often below −15° C., and in particular, alkanes and/or halogenated hydrocarbons. This class of propellant is usually liquefied at the pressure in the aerosol canister and evaporates to generate the pressure to expel the composition out of the canister. Examples of suitable alkanes include particularly propane, butane or isobutane. The second class of propellant comprises a very volatile ether of which the most widely employed ether hitherto is dimethyl ether. This propellant can advantageously be employed at relatively low weight ratio of propellant to base formulation, for example to as low as 5:95. It can also be employed in admixture with, for example, compressible/liquefiable alkane gasses. The third class of propellant comprises compressed non-oxidising gasses, and in particular carbon dioxide or nitrogen. Inert gases like neon are a theoretical alternative.

When the composition of the invention is delivered in a roll-on, a firm solid or a stick format, the topically acceptable carrier comprises a hydrophobic carrier or an aqueous carrier. The hydrophobic carrier in such cases may comprise a silicone compound, low boiling alcohol or a wax. When the composition comprises a propellant it is delivered as an aerosol.

The composition of the invention preferably additionally comprises a fragrance. By a fragrance is meant a molecule or a composition comprising a group of molecules that produces a pleasant odour. The composition preferably comprises a fragrance in 0.1 to 3% by weight of the composition.

The composition of the present invention can comprise a wide range of other optional components. The CTFA Personal care Ingredient Handbook, Second Edition, 1992, which is incorporated by reference herein in its entirety, describes a wide variety of non-limiting personal care and pharmaceutical ingredients commonly used in the skin care industry, which are suitable for use in the compositions of the present invention. Examples include: binders, biological additives, buffering agents, colorants, thickeners, polymers, astringents, fragrance, conditioners, exfoliating agents, pH adjusters, preservatives, natural extracts, essential oils, skin sensates, skin soothing agents, and skin healing agents.

According to another aspect of the present invention there is provided a method of controlling or eradicating S. hominis from skin comprising the step of applying a composition of the invention on to a desired skin surface. The method is preferably non-therapeutic. The method is especially useful for reducing or eliminating malodour especially axillary malodour.

The invention will now be illustrated with the help of the following non-limiting examples.

### EXAMPLES

### Examples 1 and 2: Efficacy of Purified StB12 Phage-Derived Recombinant Endolysin Against S. hominis 27844 and Against S. epidermidis 12228.

Purified endolysin was dialyzed with gradient of Urea (stepwise from 6M, 4M & 2M) followed by Sodium acetate and then buffer exchanged to HEPES buffer (pH 7.0). The purified endolysin was stored in HEPES buffer and activity was evaluated against S. hominis 27844 and S. epidermidis 12228 strains. Turbidity reduction assay (TRA) was performed and contact kill assay was carried out to determine the specificity and efficacy of recombinant endolysin.

There was complete loss of turbidity with 40 μg/ml of endolysin when incubated with 0.4 OD600 culture of S. hominis at 37° C. with periodic OD measurement. Similarly, higher than 5 log reduction in S. hominis counts was observed when incubated with endolysin for 5 hours. This clearly demonstrates the lytic activity of refolded endolysin against S. hominis.

Simultaneously, to evaluate the specificity of StB12 endolysin, TRA and contact kill were performed with S. epidermidis 12228. It was observed that there was marginal reduction in turbidity with S. epidermidis when incubated with StB12 endolysin. Similarly, in contact kill experiment, about 1.5 log reduction was observed when incubated with StB12 endolysin. This clearly indicates that, StB12 endolysin is differentially active against S. hominis in comparison with S. epidermidis. The data is summarized in the table 1 below:

TABLE 1

| | | Log | Log sample | |
|---|---|---|---|---|
| Example | Sample | Control | remaining | Log kill |
| Example 1 | S. hominis 27844 | 5.43 | 0.50 | 4.93 |
| Example 2 | S. epidermidis 12228 | 5.23 | 3.68 | 1.55 |

### Examples 3-8: Efficacy of Purified StB12 Endolysin Against Clinical Isolates (Isolated From Human Volunteers)

To further evaluate the efficacy of StB12 endolysin against clinical isolates of S. hominis and S. epidermidis, contact kill assay was used to determine the specificity and efficacy of recombinant endolysin against a battery of clinical isolates.

Differential lytic efficacy of StB12 endolysin against clinical isolates of S. hominis & S. epidermidis strains in contact kill assay.

As summarized in Table-2 below, four different clinical isolates of S. hominis and two isolates of S. epidermidis strains were subjected to differential lytic efficacy using StB12 endolysin in contact kill assay. CI refers to clinical isolate coded numerically.

TABLE 2

| Example | Sample | Log kill |
|---|---|---|
| Example 3 | S. hominis CI1 | 2.99 |
| Example 4 | S. hominis CI2 | 1.92 |
| Example 5 | S. hominis CI3 | 2.04 |
| Example 6 | S. hominis CI4 | 1.22 |
| Example 7 | S. epidermidis CI1 | 0.67 |
| Example 8 | S. epidermidis CI2 | 0.63 |

Between S. hominis and S. epidermidis clinical isolates, StB12 endolysin showed differential kill, with preference for S. hominis over S. epidermidis. Data in Table 2 above indicates that clinical isolates of S. hominis were eradicated up to 3 log kill, which is significantly higher as compared to kill of S. epidermidis clinical isolates (less than 1 log kill).

### Examples 9-11: Malodour Reduction Assay

In these experiments, it was evaluated whether log kill efficiency of StB12 endolysin translates into functional benefit viz. malodour reduction. An in-house assay which evaluates the malodour reduction efficacy of active/s by leveraging H₂S production by S. hominis and output as measured in terms of Lead sulphite production (Black precipitation) when H₂S reacts with Lead acetate.

Briefly, 1% Lead acetate solution was made in distilled water. Whatman filter paper was taken and dipped in the lead acetate solution. The excess solution was drained and allowed to dry in Laminar Air Flow for 30minutes. Once dried, the paper was wrapped in aluminum foil and autoclaved for future use. 0.3 OD culture of S. hominis 27844 was prepared in HEPES buffer (pH 7.0). The culture was serially diluted corresponding to 10⁷ & 10⁶ cells approximately. The OD adjusted bacterial cells were mixed with 48 μg/ml of STB12 endolysin in 1 ml reaction volume with cells and incubated for 5 hours @ 37° C. Post incubation, 500 μl of reaction mix was mixed with 500 μl of TSB broth with 0.1% L-cysteine in 24-well plate. 10 ppm of Ag-DTPA was used as positive control. Lead acetate paper was placed on top of plate and plate was sealed and incubated @ 37° C. overnight. The colour of the lead acetate paper was measured using LAB quantification. LAB colour space is a natural outgrowth of understanding the function of opponency in human vision. It's comprised of three axes: L represents darkness to lightness, with values ranging from 0 to 100; A represents greenness to redness with values of −128 to +127; and B represents blueness to yellowness also with values from −128 to +127. LAB measurement (in duplicates) was done for control and treated cells and the ΔE values were calculate using the following formula:

ΔE=√((L₁−L₂)²+(a₁−a₂)²+(b₁−b₂)² where L₁, a₁, b₁ are the LAB values for the first spot and L₂, a₂, b₂ are the LAB values for the second spot.

Higher the ΔE value, the darker the colour and higher the malodour.

The data is summarised in Table-3 below:

TABLE 3

| Example | Sample | Active | ΔE control | ΔE treated |
|---|---|---|---|---|
| Example 9 | 10⁶ of S. hominis | 40 mg/ml of | 33.8 | 4.5 |
| | | STB12 | | |
| Example 10 | 105 of S. hominis | 40 mg/ml of | 13.9 | 3.6 |
| | | STB12 | | |
| Example 11 | 10⁴ of S. hominis | 40 mg/ml of | 13.6 | 5.5 |
| | | STB12 | | |
| Positive control | 10⁶ of S. hominis | 10 mM Ag- | 53.5 | 0.5 |
| | | DTPA | | |

The data in the table-3 above indicates that while the positive control (Ag-DTPA) gives almost no malodour formation, the samples treated with the endolysin of the invention are highly significant in reducing malodour as compared to control samples.

### Examples 12-15: Efficacy of Purified StB12 Endolysin Against S. aureus Strains

To evaluate the efficacy of StB12 endolysin against a type strain and a clinical isolate of S. aureus, contact kill assay was used to determine the specificity and efficacy of recombinant endolysin against these strains.

As summarized in Table-4 below, two different clinical isolates of S. aureus strains were subjected to differential lytic efficacy using StB12 endolysin in contact kill assay and the effect of the endolysin is shown below:

TABLE 4

| | | Log | Relative log |
|---|---|---|---|
| Example | Sample | (CFU/ml) | reduction |
| Example 12 | Untreated S. aureus 6538 | 7.10 | — |
| | (Type Strain) | | |
| Example 13 | StB12 treated S. aureus 6538 | 6.36 | 0.74 |
| | (Type Strain) | | |
| Example 14 | Untreated S. aureus 9200 | 7.33 | — |
| | (Clinical isolate) | | |
| Example 15 | StB12 treated S. aureus 9200 | 6.81 | 0.52 |
| | (Clinical isolate) | | |

Data in Table 4 above indicates that the phage derived endolysins of the present invention are not very effective against different types of S. aureus strains as is evident from the relative log kill which is less than 1.

1. A non-therapeutic method of controlling or eradicating S. hominis from skin for malodour reduction comprising the step of:
applying, to a desired skin surface, an antimicrobial composition for specifically targeting S. hominis bacteria comprising:
(i) S. hominis phage-derived endolysins or nucleic acid molecules encoding the same; and
(ii) a topically acceptable carrier.

2. The method according to claim 1 wherein said endolysin is recombinant form of S. hominis phage endolysin.

3. The method according to claim 1 wherein said endolysin is cloned from endolysin gene sequence (Gene ID: 26066873; 1467 nucleotide base pair long, 489 amino acids, protein ID: YP_009130751.1) from Staphylococcal phage StB12 (GenBank: NC_020490.2) which is codon optimized for expression in E. coli and cloned into commercially available pET303/CT-His expression vector.

4. The method according to claim 3 wherein a stop codon was removed from the 3′ end of the gene to accommodate a 6× Histidine tag.

5. The method according to claim 1 wherein nucleotide sequence of endolysin is SEQ ID NO: 1:
ATGCTGATGACCCGCAAACAGGCGGAAAAATGGCTGGATAACAGCGAAGG
CCGCCAGTATAACGCGGATGGCTATTATGGCTTTCAGTGCTATGATTATA
GCAAAATGTATTTTTATGTGGTGACCGGCGAATGGATTGGCGGCCTGAAA
GCGAGCAACATTCCGTTTGATAACAAAGCGAAAATTGAAAAATATGCGAC
CATTATTAAAAACTATGATAGCTTTCTGCCGCAGAAAGGCGATATTGTGT
GCTTTCCGAACAAATATGGCGGCGGCTATGGCCATACCGCGGTGGTGACC
AAAGCGACCCTGACCCAGTTTGAAGTGCTGGAACAGAACTGGTTTGGCAA
CGGCTGGACCGATGGCGTGGTGAAACCGGGCTGGGGCCCGGAAACCGTGA
GCCGCCGCTGGCATTATTATGATAACCCGATGTATTTTATTCGCTTTAAC
TTTCCGAAAAACGTGAACGTGGTGAAAAAAGCGAAACGCAAACTGAGCAG
CAACAAAGCGAGCGGCCAGATTAAACGCAAAAAAATTATGATTGTGGCGG
GCCATGGCTATAACGATCCGGGCGCGGTGGGCAACGGCACCAACGAACGC
GATTTTATTCGCAAAAACCTGACCCCGAAAATTGCGAACTATCTGCGCAA
AACCGGCCATGAAGTGGCGCTGTATGGCGGCAGCAGCCAGAGCCAGGATA
TGTATCAGGATACCGCGTATGGCGTGCGCGTGGGCAACAAACGCGATTAT
GGCATGTATTGGGTGAACAAACAGAACTATGATCTGATTGTGGAATTTCA
TCTGGATGCGGCGGGCGCGAGCGCGAGCGGCGGCCATGTGATTATTAGCA
GCGCGTTTAACGCGGATAGCATTGATAAAGATATTCAGGAAGTGATTAAA
GAAAACCTGGGCCAGATTCGCGGCATTACCAAACGCAGCGATCTGCTGCA
TGCGAACGTGAGCGCGGAAATTAACATGAACTATCGCCTGGCGGAACTGG
GCTTTATTACCAACAAAGAAGATATGGATTGGATTAAAAAAAACAGCGAT
AAATATGCGAAACTGATTGCGGGCGCGATTCATGGCAGCCCGATTGGCGG
CGTGGTGGCGAGCAAAAAAAAAAGCAGCAGCAAAAAACTGAACGTGCCGA
AAACCATTCCGAGCGGCTATAAACTGAACAACAAAGGCGTGCCGTATAAA
AAAGAAAAAAGCCGCTATACCGTGACCACCATTAAAGGCAACAACGTGCG
CACCACCTATAGCGATAAAAGCGAAATTACCGGCACCCTGCCGAACGGCG
AAGAAATTATTTATGATGGCGCGTTTGCGGTGAACGGCTATCGCTGGATT
ACCTATCTGAACAACGATCTGCAGCGCCGCTATATTGCGACCGGCGAAAT
TGATGAAAACGGCAAACGCACCAGCAGCTATGGCAAATTTAGCCGCGTGC
ATCATCATCATCATCAT.

6. The method according to claim 1 wherein the nucleic acid molecules comprise fragments, variants and fusions of the endolysin which are capable of specifically binding to and/or lysing cells of S. hominis.

7. The method according to claim 1 wherein the topically acceptable carrier comprises an anhydrous base, liquid, lotion, cream, foam, scrub, gel, emulsion or a propellant.

8. The method according to claim 1 in which the composition is delivered in a stick form and through a roll-on device or using a propellant containing aerosol can.

9. The method according to claim 1 in which the composition additionally comprises a fragrance.

10. The method according to claim 9 wherein the fragrance is present in 0.1 to 3% by weight of the composition.

11. (canceled)

12. (canceled)

13. The method according to claim 1 for the reduction of axillary malodour.

14. A method of non-therapeutic treatment for a human in need thereof of S. hominis phage-derived endolysins or nucleic acid molecules encoding the same for controlling or eradicating S. hominis from skin by topical application of an antimicrobial composition comprising the S. hominis phage-derived endolysins or nucleic acid molecules encoding the same and a topically acceptable carrier to a desired skin surface.

15. The method according to claim 14 for reduction or elimination of malodour.

16. The method according to claim 15 for the reduction or the elimination of axillary malodour.

17. The method according to claim 15, for the reduction or the elimination of malodour from the skin surface while maintaining natural microbiome on the skin surface.


SEQUENCE LISTING (1 sequence)

SEQ ID NO: 1 - DNA, 1467 bp, Staphylococcus phage StB12
atgctgatgacccgcaaacaggcggaaaaatggctggataacagcgaaggccgccagtataacgcggatggctattatggctttcagtgctatgattatagcaaaatgtatttttatgtggtgaccggcgaatggattggcggcctgaaagcgagcaacattccgtttgataacaaagcgaaaattgaaaaatatgcgaccattattaaaaactatgatagctttctgccgcagaaaggcgatattgtgtgctttccgaacaaatatggcggcggctatggccataccgcggtggtgaccaaagcgaccctgacccagtttgaagtgctggaacagaactggtttggcaacggctggaccgatggcgtggtgaaaccgggctggggcccggaaaccgtgagccgccgctggcattattatgataacccgatgtattttattcgctttaactttccgaaaaacgtgaacgtggtgaaaaaagcgaaacgcaaactgagcagcaacaaagcgagcggccagattaaacgcaaaaaaattatgattgtggcgggccatggctataacgatccgggcgcggtgggcaacggcaccaacgaacgcgattttattcgcaaaaacctgaccccgaaaattgcgaactatctgcgcaaaaccggccatgaagtggcgctgtatggcggcagcagccagagccaggatatgtatcaggataccgcgtatggcgtgcgcgtgggcaacaaacgcgattatggcatgtattgggtgaacaaacagaactatgatctgattgtggaatttcatctggatgcggcgggcgcgagcgcgagcggcggccatgtgattattagcagcgcgtttaacgcggatagcattgataaagatattcaggaagtgattaaagaaaacctgggccagattcgcggcattaccaaacgcagcgatctgctgcatgcgaacgtgagcgcggaaattaacatgaactatcgcctggcggaactgggctttattaccaacaaagaagatatggattggattaaaaaaaacagcgataaatatgcgaaactgattgcgggcgcgattcatggcagcccgattggcggcgtggtggcgagcaaaaaaaaaagcagcagcaaaaaactgaacgtgccgaaaaccattccgagcggctataaactgaacaacaaaggcgtgccgtataaaaaagaaaaaagccgctataccgtgaccaccattaaaggcaacaacgtgcgcaccacctatagcgataaaagcgaaattaccggcaccctgccgaacggcgaagaaattatttatgatggcgcgtttgcggtgaacggctatcgctggattacctatctgaacaacgatctgcagcgccgctatattgcgaccggcgaaattgatgaaaacggcaaacgcaccagcagctatggcaaatttagccgcgtgcatcatcatcatcatcat